(19) United States
(12) Patent Application Publication
(10) Pub. No.: US 2005/0288594 A1
(43) Pub. Date: Dec. 29, 2005

(54) Methods, device and system for in vivo diagnosis

(76) Inventors: Shlomo Lewkowicz, Kiryat Tivon (IL); Gavriel Meron, Petach Tikya (IL)

Correspondence Address: PEARL COHEN ZEDEK, LLP, 10 ROCKEFELLER PLAZA, SUITE 1001, NEW YORK, NY, 10020, US

(21) Appl. No.: 10/536,982

(22) PCT Filed: Nov. 30, 2003
(86) PCT No.: PCT/IL03/01015

§ 371 Date: May 31, 2005

(30) Foreign Application Priority Data
Nov. 29, 2002 (US) 60429564

(51) Int. Cl. A61B 6/00

(52) U.S. Cl. 600/478

(57) ABSTRACT

A system and method for in vivo diagnosis are provided. A composition consisting of at least a marking agent and a pharmaceutically acceptable carrier is administered to a patient and an autonomous in vivo device, which includes an illumination source and an image sensor, is used to obtain endo luminal images of the patient.

### FIELD OF THE INVENTION

The present invention relates to in vivo diagnosis. More specifically, the invention relates to optically based methods, devices and systems for detection of pathologies or other medical conditions in body lumens, for example, in the gastrointestinal (GI) tract.

### BACKGROUND OF THE INVENTION

Pathologies of the GI tract (which may consist for example of the pharynx, esophagus, stomach, duodenum, small bowel, and colon), include, among others, esophageal carcinoma (e.g., Barrett's esophagus), peptic ulcer diseases, colorectal carcinoma and inflammatory bowel diseases (e.g., ulcerative colitis and Crohn's disease). Early pancreatic cancer may manifest itself through neoplasms released into the small intestine by the pancreas. Gastric cancer is a major cause of death worldwide especially in developing countries. The major type of gastric cancer is adenocarcinoma, which can be further categorized into an intestinal type and a diffuse type. Intestinal type lesions are frequently ulcerative and occur in the distal stomach more often than the diffuse type. These lesions are associated with a worse prognosis than the intestinal type. Colon and rectal cancers accounts for approximately 20% of all deaths due to malignant disease in the United States. Also, cancer of the pancreas is considered the fifth leading cause of cancer deaths in the United States.

Delayed detection of GI tract cancers is a major factor contributing to an overall poor prognosis. Signs of early stages of GI tract cancers may be vague and nonspecific. The deep anatomical location of organs and parts of the GI tract may also add to the low yield of early detection. Often, gastrointestinal neoplasms arise in premalignant lesions which are only partially accessible or visible by regular endoscopy or laparoscopy.

Among known modalities used to distinguish normal from malignant tissue, are optically based techniques such as photodynamic diagnosis (PPD), also known as fluorescence diagnosis, and vital staining techniques, such as chromoendoscopy. These techniques are based on specific accumulation of administered agents, such as photosensitizers (in PPD) or pigments (in chromoendoscopy). Photosensitizers (PS) typically selectively concentrate in tumor cells, remaining inactive until exposed to light of a specific wavelength. When light of the specific wavelength is delivered to the PS containing cells, it causes fluorescence of the PS. Vital stains identify specific epithelial cells or cellular constituents by preferential diffusion, absorption or adhesion across the cell membrane.

These techniques are used in various medical fields. PDD, for example, is an established diagnostic tool in urology for the visualization of bladder lesions. In gastroenterology the technique is used to distinguish between low grade and high grade dysplasias and precancerous lesions for example, in Barrett's esophagus and in the colon. Chromoendoscopy is also used mainly for detection of Barrett's esophagus. In these cases the tissues in the different body lumens are examined by an endoscope.

Endoscopic examinations are typically expensive and stressful for patients, leading to very low patient compliance and to low yield of early detection. Additionally, a large part of the GI tract (for example, most of the small intestine) is inacessible to endoscopes. Thus, endoscopic examinations provide only a partial answer to the needs of early detection and they are not perceived as beneficial in wide screening for cancers.

There is thus a need for a patient friendly diagnostic tool capable of screening even the difficult to reach parts of body lumens, for early signs of cancer.

### SUMMARY OF THE INVENTION

According to embodiments of the invention optically based techniques are used to facilitate the difference between normal and pathological (e.g., malignant) cells in a body lumen. Embodiments of the invention relate to a typically non-invasive autonomous ingestible device, which enables in situ visualization and detection of, for example, neoplastic or malignant or damaged cells or tissue even in areas that are inaccessible to endoscopes.

In one embodiment, a system is provided for diagnosing malignancy in the GI tract. The system, according to one embodiment, includes a composition comprising a marking agent and a pharmaceutically acceptable carrier. Also included in the system is an ingestible device capable of illuminating an in vivo tissue and capable of detecting remitted light from the in vivo tissue. According to an embodiment of the invention the in vivo device may include, for example, a transmitter, typically a wireless transmitter, for transmitting data to a receiving unit that is located externally to the patient.

According to embodiments of the invention a marking agent may include, for example, molecules or compounds that selectively and/or differentially adhere to dysplasias in endo luminal tissues. According to another embodiment a marking agent may include targeting agents, such as antibodies. According to one embodiment the targeting agents are appended to a dye.

A method for detection of dysplasias and/or neoplasms and/or malignancies in the GI tract, according to an embodiment of the invention, includes the step of administering to a person a marking agent, typically by administering a composition comprising a marking agent and a pharmaceutically acceptable carrier, and the step of illuminating the GI tract walls and detecting remitted light. Typically this is performed by inserting to a body lumen, e.g., the GI tract, typically by swallowing, a device for illuminating the GI tract walls and for detecting remitted light. According to one embodiment the device may be an in vivo imaging capsule. According to some embodiments of the invention a patient may be subject to preparation procedures and/or patient positioning procedures.

### BRIEF DESCRIPTION OF THE DRAWINGS

FIG. 1 is a block diagram schematically illustrating a system according to an embodiment of the invention;

FIG. 2 shows a schematic illustration of an in vivo diagnosing device according to an embodiment of the invention; and

FIGS. 3A and 3B are flowcharts depicting methods of detecting pathologies or conditions, according to different embodiments of the invention.

### DETAILED DESCRIPTION OF THE INVENTION

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

Embodiments of the invention are based on specifically staining, for example, neoplastic, malignant or damaged cells or tissue. Other cells or conditions may also be stained according to embodiments of the invention. For example, the term “damaged cells or tissue” may include, for example, infectious or inflammatory lesions, a non-tumorous or noninfectious inflammation, clots, hyperplasia, atherosclerotic plaques and other pathologies.

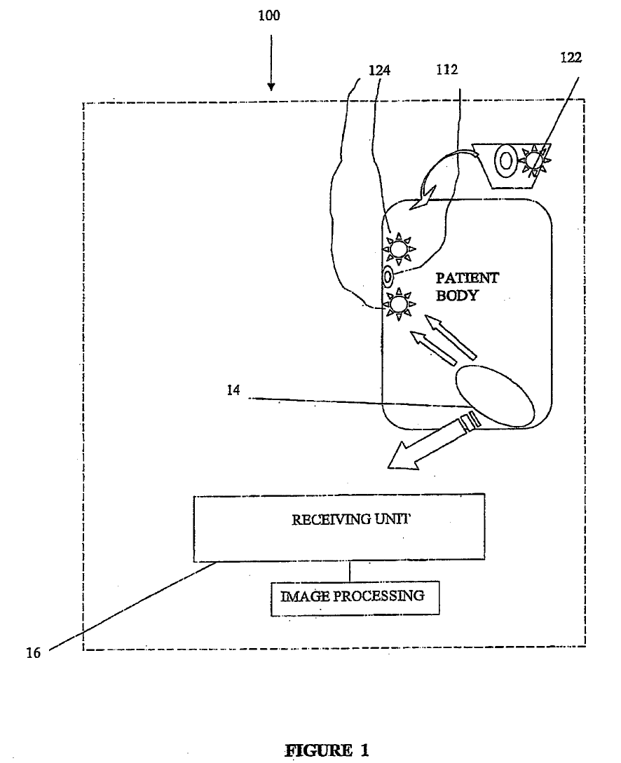

A system for in vivo diagnosis, according to an embodiment of the invention is schematically illustrated in FIG. 1. The system 100 typically includes a marking agent 12, an in vivo light detector, such as imaging device 14 and a receiving unit 16, typically for receiving image information. The marking agent 12 is typically contained in a composition 122, which comprises a pharmaceutically acceptable carrier 124. According to an embodiment of the invention the composition 122 is introduced into a patient's body lumen in which the marking agent 12 (and possibly the carrier 124) typically migrate to the lumen wall. The imaging device 14 is also introduced into the patient's body lumen and images of the body lumen environment may be obtained. The obtained image data is transmitted to a typically external receiving unit 16 for, according to some embodiments, further analysis and diagnosis. Receiving unit 16 may include image processing capabilities. According to some embodiments image processing takes place either prior to or after the image data is transmitted to the receiving unit. According to some embodiments other light detectors may be used, such as photoelectric cells.

The marking agent 12 typically contains molecules or compounds that may be optically visible and which selectively and/or differentially adhere to dysplasias in endo luminal tissues. The term dysplasias may be understood to include, inter alia, neoplasms, lesions and/or malignancies.

According to one embodiment, marking agent 12 includes photosensitizers such as 5 aminolevulinic acid (5-ALA). Photosensitizers can be activated by light of a specific wavelength to emit light of a different wavelength., 5-ALA, for example, is a natural precursor of the heme biosynthetic pathway and it induces formation of protoporphyrinIX (PPIX), an endogenous photosensitizer. Application of 5-ALA leads to intracellular accumulation of endogenous PPIX in areas of high grade dysplasias and malignant lesions.

According to another embodiment marking agent 12 may include substances known as vital stains, such as, for example, methylene blue, indigo carmine, Lugol iodine and toluidine blue. Vital stains may include, for example, absorptive stains, which typically identify specific epithelial cells or cellular constituents by preferential diffusion or absorption across cell membranes, and contrast staining which typically highlights tissue topography accentuating irregularities in surface contours. A pharmaceutically acceptable carrier 124 may include, for example, high molecular weight molecules that are typically not absorbed by the stomach. These may include, for example, gelatinous substances and/or other proteins. According to yet another embodiment the marking agent 12 may include, for example, tumor marker targeted molecules. Tumor markers are molecules occurring in blood or tissue that are associated with cancer. Typically, tumor markers are products of cancerous cells or the cells themselves and they represent aberrant production of what may be a typically normal element. Some markers are produced in response to the presence of cancer, such as antibodies. Tumor marker targeted molecules typically have a high affinity to tumor markers and, under certain conditions, will adhere to tumor markers in a liquid environment These may include, for example, antigens having specificity to tumor marker antibodies. Alternatively, tumor marker targeted molecules may include, for example, antibodies specific to tumor marker antigens. According to an embodiment of the invention a tumor marker targeted molecule may be modified to include additional markers such as a dye or a radioactive or fluorescent moiety.

The composition 122 may be in the form of a powder, tablet, pill, suspension, liquid, spray or any other suitable form for administering the marking agent 12 to the endo luminal walls.

According to an embodiment of the invention the in vivo imaging device 14 may be an ingestible capsule which may include an illumination source (not shown) for illuminating a body lumen, typically the GI tract, an image sensor (not shown) for obtaining images of the body lumen and a transmitter (not shown) for transmitting image or other data to the receiving unit 16. Receiving unit 16 is typically located outside a patient's body and may include an antenna or antenna array (not shown), for receiving image and possibly other data from the device 14, a receiver storage unit, for storing image and other data, a data processor, a data processor storage unit, and an image monitor (not shown), for displaying, inter alia, the images transmitted by the device 14 and recorded by the receiving unit 16. Typically, the receiver and receiver storage unit are small and portable, and may be worn on the patient's body during recording of the images. Typically, data processor, data processor storage unit and monitor are part of a personal computer or workstation, which may include standard components such as a processor, a memory, a disk drive, and input-output devices, although alternate configurations are possible. In alternate embodiments, the data reception and storage components may be of another configuration. Processing capabilities, for example for processing real image data and fluorescent data, may be included in the receiving unit or in the workstation.

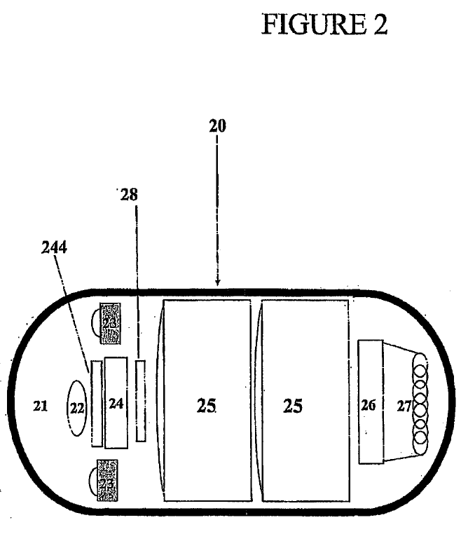

Reference is now made to FIG. 2, which schematically illustrates an in vivo diagnosing device, such as device 14 of FIG. 1, according to an embodiment of the invention. In an exemplary embodiment, the device 20 is an autonomous capsule shaped device capturing images of a body lumen, typically of the GI tract. However, the device 20 may be of other suitable shapes. Typically, device 20 includes at least one sensor or light detector such as an image sensor 24, for capturing images and at least one illumination source 23 (two illumination sources 23 are included in FIG. 2 for illustrative purposes). The image sensor 24 may be a CMOS, CCD or any other suitable in vivo image sensor. An optical dome 21 provides a generally transparent cover for the optical elements, provides a sealed barrier to bodily fluids, and may perform other functions (such as holding optical elements). An optical system 22, including, for example, one or more optical elements, such as one or more lenses or composite lens assemblies, or any other suitable optical elements, may aid in focusing reflected light onto the image sensor 24 and performing other light processing. The device 20 may include a dedicated light detector, such as photodetector 28 and/or a filter 224 which may cover all or some of the pixels of the image sensor 24. Device 20 typically includes a transmitter 26, for transmitting image and possibly other (e.g., non-image) information to a receiving device, and may include other components, such as, for example, a compression module for compressing data. According to one embodiment the transmitter 26 is an ultra low power radio frequency (RF) transmitter with high bandwidth input, possibly provided in chip scale packaging. The transmitter 26 may transmit via an antenna 27. The transmitter 26 may also include circuitry and functionality for controlling the device 20. For example, a master clock included in transmitter 26 may control illumination functions of the device. Typically, the device includes a power source 25, such as one or more batteries. For example, the power source 25 may include chargeable batteries, silver oxide batteries, lithium batteries, or other electrochemical cells having a high energy density, or the like. Other, typically internal power sources may be used. Other components and sets of components may be used. For example, the power source may be an external power source transmitting power to the capsule, and a controller separate from the transmitter 26 may be used. Examples of in-vivo devices and systems that may be used with the present invention are described in U.S. Pat. No. 5,604,531 to Iddan entitled “An In-vivo Camera Video System” and/or in International Application Publication No. WO 01/65995, entitled “A Device and System for In-Vivo Imaging”, both of which are assigned to the common assignee of the present invention and are hereby incorporated herein by reference. Further, other devices and systems for in-vivo imaging may be used, having imaging devices and reception/display systems of other configurations.

According to one embodiment fluorescent dyes are utilized to specifically stain neoplastic and/or malignant cells. Accordingly, the in vivo device 20 may include a plurality of light sources, for example, a polychromatic (e.g., white) light source for obtaining real images and a monochromatic (e.g., blue) light source for illuminating (energizing) fluorescent dyes. According to an embodiment of the invention photosensitizing drugs are administered to a patient and after a metabolic period but prior to decline of sensitivity, an imaging device is ingested for obtaining images of practically the entire GI tract. Imaging of the GI tract may be done in a regular mode, in which images are obtained by illuminating typically white light. Imaging may also be done in a fluorescent mode in which light is illuminated in a first wavelength and remitted light of a second wavelength is collected. According to some embodiments an illumination source may be activated in a flashing mode, having alternating light and dark periods. Thus, for example, embodiment endo—luminal sites may be illuminated by white light followed by a dark period in which fluorescent emission may be detected. According to yet another embodiment dyes, such as vital stains, are utilized to specifically or differentially stain neoplastic and/or malignant cells. In this embodiment imaging in a regular mode may provide optical information for diagnosing the endo-luminal condition.

Referring back to FIG. 2, according to one embodiment, device 20 is swallowed by a patient to whom a photosensitizer or another dye has been administered. Device 20 is swallowed about 4-6 hours after the administration of a photosensitizer or within about 10 minutes after application of a dye such as a vital stain onto the GI tract wall. As the device 20 is swallowed it is activated and illumination source 23, which may be for example a white LED begins to illuminate. Remitted light is focused through optical system 22 onto image sensor 24, or, optionally onto photodetector 28, thereby forming images that are then transmitted by transmitter 26. According to another embodiment activation of the device 20 causes the successive action of a white LED and a monochromatic LED (e.g., a blue LED), for obtaining a real image and a successive fluorescent image. The two images may then be processed, for example by image subtraction, to obtain diagnostic information.

According to one embodiment a patient is administered photosensitizer, such as 5-ALA, which induces the formation of PPIX. PPIX can be induced by violet light to emit red light. In this case device 20 may include at least one illumination source 23 which is a monochromatic light source, for example, in the range of about 400 nm. In alternate embodiments light source 23 may include a filter (not shown) such as a bandpass filter for obtaining violet-blue illumination. Capsule 20 may be swallowed by a patient to whom 5-ALA has been administered, and light source 23 may illuminate in violet whereas remitted light in the range of 600-800 nm will be detected by dedicated photodetector 28, which may be any light detecting device such as a CCD camera or by image sensor 24 due to filter 244 that may be any suitable filter, such as a red filter. In another embodiment illumination source 23 may be controlled (for example by transmitter 26) to illuminate in a flash mode where each flash of light is followed by a dark period. White light or monochromatic light may be used to illuminate the GI tract wall whereas real image frames may be obtained during the illumination period and fluorescent images may be obtained during the dark periods. Any image obtained during the dark period is a result of the delayed fluorescent emission of a photosensitizer or other fluorescent dye. Data obtained from fluorescent images may be indicative of dysplasias or malignancies.

According to another embodiment images of the GI tract wall obtained from a patient to whom a dye, such as methylene blue, has been administered, will be stained blue.

Teal images of the stained GI tract wall may provide data regarding the staining. Differential staining, for example, absent or inhomogeneous staining in the case of methylene blue, targets possible dysplasia or cancer.

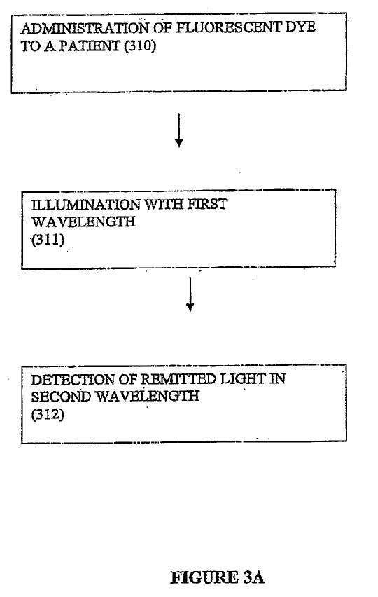

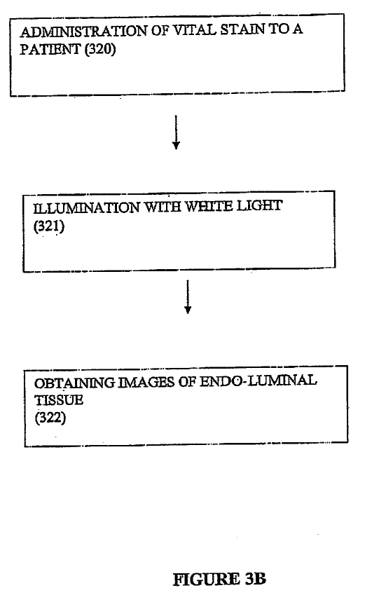

Methods for detecting, for example, malignant and/or damaged cells or tissue, according to different embodiments of the invention are depicted in FIGS. 3A and 3B.

According to one embodiment (FIG. 3A) a fluorescent dye is administered to a patient (310). The body lumen wall is then illuminated in a first wavelength (311) and remitted light in a second wavelength is then detected (312). Images of the remitted light in the second wavelength may then be used to diagnose dysplasias of the lumen wall.

A fluorescent dye may be a photosensitizer, for example, as described above. According to another embodiment a fluorescent dye may be a tumor marker targeted dye complex. According to some embodiments tumor marker targeted molecules may include, for example, antibodies to antigenic determinants associated with cancer, such as, CA199 (an antigenic determinant associated with cancers such as pancreas, colorectal and gastric) and CEA (an antigenic determinant associated with cancers such as pancreas, colorectal, liver and gastric). According to other embodiments tumor marker targeted molecules may include, for example, antigenic determinants having affinity to antibodies associated with cancer, such as Gastric Mucin, which is specific to an IgG antibody associated with stomach and colon cancers. Many tumor marker targeted molecules are commercially available, for example, 116-NS-19-9 is a CA199 specific antibody and mCEA is a CEA specific antibody, both available from ARUP Laboratories of the Fitzgerald Industries. These molecules may be modified as is known in the art to include a dye moiety. According to some embodiments, through formation of an antibody-antigen complex, a tumor marker targeted molecule is linked to a target cancer cell and will adhere to that cell even after washing of the tissue. A tumor marker targeted molecule carrying a fluorescent dye may then be imaged by being illuminated with a first wavelength and emitting a second wavelength which may be detected, for example, by using appropriate color filters or dedicated photodetectors.

Typically, the marker agents, such as the dyes or target molecules containing dyes, are contained in a pharmaceutical composition. The composition may be formulated to, for example, a tablet or a capsule, a composition for injection, a spray, an aerosol, topical composition, a syrup or any suitable form. In cases where there is more than one composition, each of the compositions can be manufactured in a different form. In another embodiment, the compositions can be manufactured in the same form. The compositions may be administered by any suitable route, for example, by oral application or by direct application during surgery using a laparoscope or trocar. In alternate embodiments a dye containing composition may be sprayed onto a lumen wall by methods known in the art, for example, by use of an endoscope or by use of burst release capsules. Tablets that release active ingredients (e.g., according to embodiments of the invention, a dye) upon changes in environmental pH or temperature are known and may be utilized according to certain embodiments of the invention.

Compositions according to embodiments of the invention may include a pharmaceutically acceptable carrier. The choice of carrier can be determine in part by the particular dye used, as well as by the particular route of administration of the composition. The carrier is typically compatible with both the dye and the tissues and organs of the patient. Moreover, the carrier typically does not interfere with the energy applied or images obtained following administration.

Other additives may be included in compositions according to embodiments of the invention. Appropriate additives may be selected such that they do not interfere with the activity of the targeted molecule and such that they may facilitate the reaction of the targeted molecule with a tumor marker. In some instances, an additive is selected to increase the specificity, toxicity, solubility, serum stability, and/or intracellular availability of the targeted moiety.

If oral administration is desired, the marking agent should be provided, for example, in a composition that protects it from the acidic environment of the stomach. For example, the composition can be formulated in an enteric coating that maintains its integrity in the stomach and releases the active compound in the intestine. Oral compositions will generally include an inert diluent or an edible carrier and may be compressed into tablets or enclosed in gelatin capsules. For the purpose of oral administration, the active compound or compounds can be incorporated with excipients and used in the form of tablets, capsules or troches. Pharmaceutically compatible binding agents and adjuvant materials can be included as part of the composition

The step of administering a marker agent to a patient may be preceded by preparation steps, which may include washing the lumen wall, for example by ingesting a volume of water. Other preparation steps may include having the patient fast prior to administration or emptying of lumens, such as the large intestine. The steps of illuminating and detecting remitted light can be performed, for example, as described above by using an in vivo imaging device. Inserting an in vivo device into body lumens may be accompanied by positioning of the patient so as to ensure proper positioning of the device in the lumen and full coverage of the lumen.

Steps of a method according to one embodiment of the invention are illustrated in the Example below.

Note that the various sequences provided are exemplary only, and are not intended to be limiting; other steps or series of steps may be used. Other ranges or amounts may be used, other diagnostic agents, and other configurations for an imaging/display device and system, if suitable.

### EXAMPLE 1

Procedure for PPD Using an Ingestible Imaging Capsule
1. A patient after a 12 hour fast is given 0.5 liter water;
2. half an hour later 5-ALA is administered to the patient in the form of a powder of more than 99% purity (commercially available, e.g. from Medac GmbH, Hamburg, Germany). A water/juice solution is prepared at a dose of 15-60 mg/Kg body weight, and is orally ingested by the patient;
3. 4-7 hours after oral ingestion the patient ingests 0.5 liter of water to wash the GI tract tissue from excess 5-ALA;
4. 10 minutes after the ingestion of water the patients ingest a Given™ capsule which includes at least one blue LED and a red filter over parts of the CMOS image sensor;
5. images of the GI tract are obtained in the usual manner (recorded and down loaded to the RAPID™ work station) as well as fluorescent images obtained by the CMOS image sensor. For view of the entire GI tract images are collected for a period of at least 8 hours;

6. Analysis of the images may include comparing white light image frames to consecutive or simultaneous fluorescent image frames.

It will be appreciated that liquids reach the stomach in a matter of minutes, the small intestine in the range of 1-3 hours and the large intestine in about 6 hours. Thus, the timing of the different steps may be adjusted as needed.

According to a second embodiment (FIG. 3B) a vital stain dye is administered to a patient (320). The body lumen wall is then illuminated typically by white light (321) and images of the lumen wall are then obtained (322). The obtained images may then be used to diagnose dysplasias of the lumen wall. Image processing (e.g., color, enhancement) capabilities of the imaging device or of its workstation may be utilized.

Vital stains that may be used according to embodiments of the invention may include, for example, methylene blue (detailed above), indigo carmine, which is a blue contrast stain and which can accentuate subtle mucosal abnormalities, Lugol iodine solution, which is an absorptive stain with an affinity for glycogen in nonkeratinized squamous epithelium. After administration of Lugol iodine solution normal esophageal mucosa turns a prominent green-brown color within moments, gradually fading over minutes to hours. In this case absence of staining indicates diminished or absent glycogen content, for example, as seen in squamous cell cancers, dysplasia, Barrett epithelium and gastric metaplasia. Toluidine blue may also be used for staining of nuclei, thereby staining tissues having increased mitotic activity.

According to one embodiment an in vivo imaging device having at least one monochromatic illumination source (for example, a blue LED) may be used for better detection of the vital stains. Alternately, color filters may be used for viewing images obtained from the GI tract, for enhancing detection of vital stains.

Steps of methods according to further embodiments of the invention are illustrated in the Examples below.

### EXAMPLE 2

Procedure I for Chromo-Capsule Endoscopy
1. 1% sterile solution of methylene blue is sprayed onto the esophagus mocosa;
2. 10% acetylcysteine (mucolytic agent) is applied to the esophagus mucosa within minutes of the methylene blue application;
3. A horizontally positioned patient immediately ingests a Given™ M2A™ capsule;
4. The patient may be slowly rotated;
5. images of the GI tract are obtained in the usual manner (recorded and down loaded to the RAPID™ work station). For view of the entire GI tract images are collected for a period of at least 8 hours.

### EXAMPLE 3

Procedure II for Chromo-Capsule Endoscopy
1. 0.5% to 0.8% solution of indigo carmine is sprayed onto the esophagus mucosa;
2. A horizontally positioned patient immediately ingests a Given™ M2A™ capsule which includes at least one blue LED;
3. images of the GI tract are obtained in the usual manner (recorded and down loaded to the RAPID™ work station). For view of the entire GI tract images are collected for a period of at least 8 hours.

It will be appreciated that the positioning and rotating of the patient may enhance positioning and coverage by the capsule of the lumen being inspected. For example, swallowing a capsule while being horizontal positioned ensures the capsule stays in the esophagus for a desired amount of time.

For efficient view of areas such as the esophagus a specifically designed imaging capsule may be used. For example, a capsule having a plurality of optical pathways may be used for obtaining a wide angle of view. Such a capsule is described, for example, in WO 02/054932 which is assigned to the common assignee of the present invention and which is hereby incorporated by reference. Alternately, careful positioning of a patient while swallowing an imaging capsule may assist in keeping the imaging capsule in a desired location for obtaining images, for example, of the esophagus.

According to some embodiments of the invention there are provided kits for diagnosing malignant or damaged cells or tissues. According to one embodiment a kit may contain at least one composition which comprises a marker agent. According to another embodiment a kit may also contain a typically single use wireless, typically autonomous in vivo imaging device.

It will be appreciated by persons skilled in the art that the present invention is not limited to what has been particularly shown and described hereinabove. Rather the scope of the present invention is defined only by the claims, which follow:

1-23. (canceled)

24. A method of imaging diagnostic information on the GI tract, the method comprising:
administering to a patient a composition comprising a fluorescent dye;
flashing illumination within the GI tract, thereby providing a light period and a dark period;
obtaining a fluorescent image of the GI tract tissue during the dark period; and
transmitting image data.

25. The method of claim 24 comprising obtaining, on an image sensor within an ingestible capsule, a real image of the tissue during the light period.

26. The method of claim 25 comprising obtaining the fluorescent image and the real image on the same image sensor.

27. The method of claim 24 wherein flashing comprises alternately illuminating with white light.

28. The method of claim 24 wherein transmitting is wireless

29. The method of claim 24 comprising focusing light remitted from the GI tract tissue onto an image sensor within an ingestible capsule.

30. The method of claim 24 wherein flashing comprises alternately illuminating with monochromatic light and white light.

31. The method of claim 25 comprising processing the real image and the fluorescent image to obtain diagnostic information.

32. A system for imaging a GI tract, the system comprising an ingestible imaging device, the device comprising:
a polychromatic illumination source;
a monochromatic illumination source;
an image sensor to receive a real image and a fluorescent image; and
a transmitter to transmit image data to an external receiving unit

33. The system of claim 32 comprising an optical system to focus light remitted from the GI tract onto the image sensor.

34. The system of claim 32 comprising a receiving unit for receiving and processing the image data.

35. A method comprising:
staining cells of an endo-luminal wall;
illuminating said cells with an in vivo illumination device from within a lumen;
capturing light remitted from said cells.

36. The method as in claim 35, wherein said illumination comprises a polychromatic illumination and a monochromatic illumination.

37. The method as in claim 35, wherein said capturing comprises capturing a real image and a fluorescent image.
